(19) United States
(12) Patent Application Publication
(10) Pub. No.: US 2022/0378357 A1
(43) Pub. Date: Dec. 1, 2022

(54) DEVICE AND METHOD FOR ACQUIRING BRAIN ELECTRICAL SIGNAL

(71) Applicant: TSINGHUA UNIVERSITY, Beijing (CN)

(72) Inventors: XIAO-RONG GAO, Beijing (CN); DONG-BING WANG, Beijing (CN); YI-KE SUN, Beijing (CN); QI-YUAN MA, Beijing (CN)

(21) Appl. No.: 17/883,629

(22) Filed: Aug. 9, 2022

Related U.S. Application Data
(63) Continuation of application No. PCT/CN2021/094637, filed on May 19, 2021.

(30) Foreign Application Priority Data
Apr. 27, 2021 (CN) 202110458653.4

(51) Int. Cl. A61B 5/369; G06F 3/01

(52) U.S. Cl. CPC A61B 5/369; G06F 3/015

(57) ABSTRACT

A device and a method for acquiring brain electrical signal. The device for acquiring brain electrical signal includes a plurality of electrodes and an electrode base. The electrode base is provided with a plurality of screw holes, and each of the plurality of electrodes passes through a corresponding screw hole of the plurality of screw holes.

### CROSS-REFERENCE TO RELATED APPLICATION

The present application is a continuation application of PCT Patent Application No. PCT/CN2021/094637, entitled “Device and Method for Acquiring Brain Electrical Signal”, filed on May 19, 2021, which claims priority to Chinese Patent Application No. 202110458653.4, entitled “Device and Method for Acquiring Brain Electrical Signal”, filed on Apr. 27, 2021, the entire contents of which are incorporated herein by reference

### TECHNICAL FIELD

The present application relates to the technical field of medical apparatus, in particular to a device and a method for acquiring brain electrical signal.

### BACKGROUND

Brain-Machine/Computer Interface (BMI/BCI) technology refers to establishing a communication and control bridge between the brain and external electronic devices, and achieving communication between the human brain and the machine by collecting and decoding neurophysiological signals. The BMI/BCI technology is not only of great significance for a diagnosis and a mechanism research of cranial nerve diseases, but also has a broad application prospect in the fields of emergency rescue, industrial production and even military science.

Existing methods for acquiring brain electrical signal are mainly divided into two types: an intrusive type and a non-intrusive type. Quality of signals, such as Electroencephalogram (EEG), acquired by existing non-intrusive electrophysiological monitoring methods, is relatively poor, and bone tissues, such as the skull, cause electrical signals to attenuate greatly. The quality of signals acquired by existing intrusive/semi-intrusive electrophysiological monitoring methods such as Electrocorticography (ECoG), is very good, but an auxiliary surgery such as a craniotomy is required, which is very risky. Except for patients with epilepsy, most people are unwilling to have a craniotomy to have electrodes implanted, which is rather disadvantageous to generalization.

### SUMMARY

Based on this, in view of the existing problems during acquisition of brain electrical signals, it is necessary to provide a device and a method for acquiring brain electrical signal.

The present application provides a device for acquiring brain electrical signal, including a plurality of electrodes, and an electrode base provided with a plurality of screw holes. Each of the plurality of electrodes passes through a corresponding screw hole of the plurality of screw holes.

In an embodiment, the device for acquiring brain electrical signal further includes an amplifier arranged on the electrode base.

In an embodiment, each of the plurality of electrodes has a first end and a second end. The first end of each of the plurality of electrode is a physiological signal monitoring end. The second end of each of the plurality of electrode is provided with a spiral wire. The second end of each of the plurality of electrode is connected to the electrode base by the spiral wire.

In an embodiment, the plurality of screw holes are arranged in an array on the electrode base.

In an embodiment, the device for acquiring brain electrical signal further includes an amplitude regulating rod, connected to the electrode base and configured to adjust a vibration amplitude.

In an embodiment, a surface of the electrode base away from the amplitude regulating rod is a round-edged surface.

In an embodiment, the device for acquiring brain electrical signal further includes a water pore, disposed in the amplitude regulating rod.

In an embodiment, the device for acquiring brain electrical signal further includes a fixing case connected to the amplitude regulating rod, and the plurality of electrodes are fixed on the fixing case.

In an embodiment, the device for acquiring brain electrical signal further includes a transducer unit, electrically connected to the plurality of electrodes, and configured to supply energy to the plurality of electrodes. The transducer is configured to generate mechanical waves, namely, ultrasound.

In an embodiment, each of the plurality of the electrodes is a threaded electrode needle.

In an embodiment, the amplifier is arranged inside the electrode base or on an outer side wall of the electrode base.

In an embodiment, the transducer unit includes: a front cap, a plurality of piezoelectric ceramics, a plurality of electrodes, a first electrode wire, a second electrode wire, and a rear cap.

The plurality of electrodes and the plurality of piezoelectric ceramics are arranged between the front cap and the rear cap.

The plurality of electrodes and the plurality of piezoelectric ceramics are electrically connected one-to-one.

Two ends of each of the plurality of electrodes are electrically connected to the first electrode wire and the second electrode wire, respectively.

In an embodiment, the transducer unit further includes a central shaft.

The plurality of electrodes are received inside the central shaft, and the central shaft is configured to run to a position of a spiral wire arranged at the second end of each of the plurality of electrodes.

The first end of each of the plurality of electrodes is an electrode needle.

In an embodiment, the fixing case, the amplitude regulating rod, the electrode base and the rear cap are insulated.

This application provides a method for acquiring brain electrical signal, including monitoring physiological signals by using any one of the above devices for acquiring brain electrical signal.

This application provides the device for acquiring brain electrical signal and the method for acquiring brain electrical signal. The device for acquiring brain electrical signal includes the plurality of electrodes configured to monitor the physiological signals, and the electrode base configured to support the plurality of electrodes. The electrode base is provided with the plurality of screw holes, and the plurality of screw holes are configured to support the plurality of electrodes. Each of the plurality of electrodes passes through a corresponding screw hole of the plurality of screw holes. By means of the plurality of electrodes, the device for acquiring brain electrical signal may be directly implanted in the bone, and may directly touch or intrude endocranium or other soft tissues, to record the electrophysiological signals. The device for acquiring brain electrical signal acquires the cortical EEG signals by means of the plurality of electrodes and the electrode base.

### BRIEF DESCRIPTION OF THE DRAWINGS

In order to illustrate the embodiments of the present application or the technical solutions in the prior art more clearly, the accompanying drawings that are needed for describing the embodiments or the prior art will be described briefly in the following description. Obviously, the drawings described herein are merely some examples of the present invention. For those of ordinary skill in the art, other drawings may be obtained according to these drawings without creative work.

FIG. 1 is a schematic structural view of a device for acquiring electroencephalogram (EEG) signal according to an embodiment of the present application.

FIG. 2 is a schematic structural view of an electrode base of the device for acquiring brain electrical signal according to an embodiment of the present application.

FIG. 3 is a schematic structural view of an electrode of the device for acquiring brain electrical signal according to an embodiment of the application.

FIG. 4 is a schematic flow chart illustrating steps of a method for acquiring brain electrical signal according to an embodiment of the application.

Illustration of reference numerals:

100. device for acquiring brain electrical signal

1. round-edged surface; 2. electrode base; 3. water pore; 4. amplitude regulating rod; 5. fixing case; 6. front cap; 7. piezoelectric ceramic;

8. electrode plate; 9. first electrode wire; 10. second electrode wire; 11. rear cap; 12. central shaft; 13. screw hole;

14. spiral wire; 15. electrode; 20. transducer unit.

### DETAILED DESCRIPTION OF THE EMBODIMENTS

In order to make the objectives, technical solutions and advantages of the present application clearer and better understood, the present application will be further described in detail herein with reference to the accompanying drawings and embodiments. It should be understood that the specific embodiments described herein are merely intended to illustrate the present application but not intended to limit the present application.

The serial numbers assigned to the components herein, such as “first”, “second”, etc., are only used to distinguish the described objects, but neither represent any sequence nor have technical meaning. The “connection” and “communication” described in the present disclosure include direct connection and indirect connection (communication) unless otherwise specified. In the description of the present application, it should be understood that the orientations or the positional relationship indicated by the terms “upper”, “lower”, “front”, “rear”, “left”, “right”, “vertical”, “horizontal”, “top”, “bottom”, “inner”, “outer”, “clockwise”, “counterclockwise”, etc., are based on the orientations or positional relationship shown in the accompanying drawings, and they are used only for the convenience of describing the application and simplifying the description, rather than indicating or implying that the related device or component must have a specific orientation, must be constructed and operated in the specific orientation, therefore these terms cannot be understood as limitation of the present application.

In the present application, unless expressly stipulated and defined otherwise, the first feature is “on” or “under” the second feature may be that the first feature is in direct contact with the second feature, or that the first feature is in indirect contact with the second feature by means of an intermediate medium. Moreover, the first feature is “over”, “above” and “on” the second feature may be that the first feature is directly above or obliquely above the second feature, or simply means that a level of the first feature is higher than that of the second feature. The first feature is “under”, “below” and “beneath” the second feature may mean that the first feature is directly below or obliquely below the second feature, or it simply means that the level of the first feature is smaller than that of the second feature.

The inventors comprehensively analyzed that, compared with the non-intrusive acquisition method, such as Electrocorticography , the intrusive signal acquisition method has unique advantages like high temporal and spatial resolution, good signal quality, and strong stability, etc. Further, according to different monitoring positions of the nerve electrodes, the intrusive signal acquisition methods include ECoG and intracortical neuron recording, and other signal acquisition methods. Although the spatial resolution of brain electrical signals acquired by ECoG, semi-intrusive brain-computer interface technology, is lower than that of the brain electrical signals acquired by intrusive neuronal potential recording, the non-intrusive method has incomparable advantages in a long-term implantation tracking and monitoring because the non-intrusive method causes few immune responses and tissue damages.

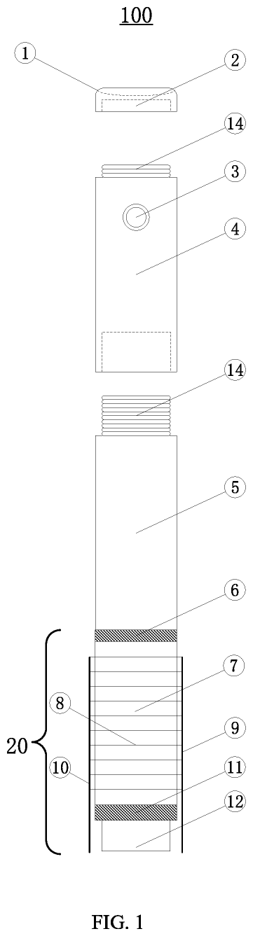

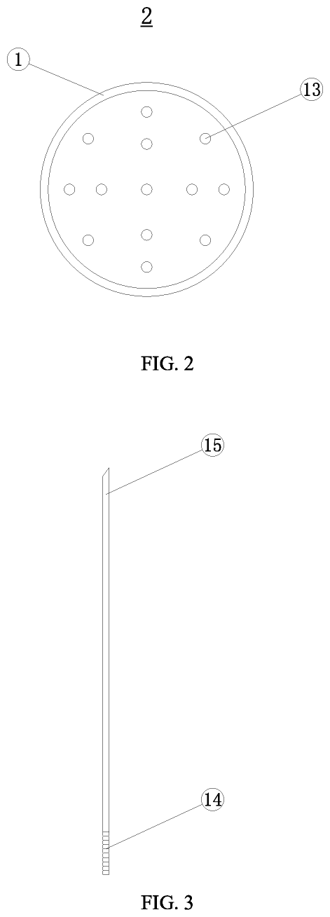

Please refer to FIGS. 1, 2 and 3, the present application provides a device for acquiring brain electrical signal 100. The device for acquiring brain electrical signal 100 provided in the present application includes a plurality of electrodes 15 and an electrode base 2.

The plurality of electrodes 15 are configured to be inserted into a living body to monitor physiological signals. The electrode base 2 is provided with a plurality of screw holes 13. The plurality of screw holes 13 are configured to support the plurality of electrodes 15. Each of the plurality of electrodes 15 passes through a corresponding screw hole of the plurality of screw holes 13. Each of the plurality of electrodes 15 passes through the corresponding screw hole of the screw holes 13, thus ensuring that the electrodes 15 to be fixed in the electrode base 2.

The device for acquiring brain electrical signal 100 is configured to monitor physiological signals, and the device for acquiring brain electrical signal 100 is inserted into the living body by means of the plurality of electrodes 15 to monitor the physiological signals. The plurality of electrodes 15 are inserted into the living body and cause small wound to the living body, and the monitoring for signals is more accurate. Specifically, in the device for acquiring brain electrical signal 100 provided in the embodiments of the present application, the plurality of electrodes 15 drill holes, and the plurality of electrodes 15 (or called drills) are directly implanted in the bone, and directly touch or intrude endocranium or other soft tissues, to record the electrophysiological signals.

In an embodiment, a material of the plurality of electrodes 15 may be at least one of metal, metal alloy, and conductive polymer. For example, the metal may be platinum, silver, iridium, nickel, or cobalt. The metal alloy may be a solid product with metallic properties obtained by mixing and melting one metal with another metal, or with several metals or non-metals, and then cooling and solidifying. For example, the metal alloy may be manganese steel, stainless steel, brass, bronze, cupronickel, solder, or duralumin. The conductive polymer may be a conductive polymer material, or a type of polymer material in which a polymer with a conjugated π-bond is chemically or electrochemically “doped” to convert itself from an insulator to a conductor. A length and a thickness of the plurality of electrodes 15 are not specifically limited herein, and may be arbitrarily adjusted according to design requirements of an ultrasonic osteotome.

The device for acquiring brain electrical signal 100 provided in this embodiment may transform electrical energy into mechanical energy, so that the plurality of electrodes 15 are in a high-frequency resonance mode, and implanted or non-implanted physiological signal monitoring for the target tissue is implemented by means of a great mechanical acceleration of the plurality of electrodes 15. In addition, the device for acquiring brain electrical signal 100 has high operational safety, may cut accurately, and will not easily damage surrounding tissues, thereby ensuring the living body in a treatment to be safer. The device for acquiring brain electrical signal 100 may be applied to some precision orthopedic surgeries. The cutting speed of the device for acquiring brain electrical signal 100 provided in the present application is moderate. When the device for acquiring brain electrical signal 100 monitors the physiological signals, it will not easily injure the soft tissues of the living body and will not cause serious sequelae just because of a minor improper operation.

In an embodiment, the device for acquiring brain electrical signal 100 further includes an amplifier (not shown in FIG. 1).

The amplifier is arranged on the electrode base 2, and configured to amplify the physiological signals monitored by the plurality of electrodes 15. Specifically, the amplifier may be arranged inside the electrode base 2 or on an outer side wall (not shown in the figure) of the electrode base 2. The specific structure of the amplifier is not limited, as long as it can perform the function of amplifying the monitored physiological signals. In this embodiment, as a base of the amplifier, the electrode base 2 of the device for acquiring brain electrical signal 100, together with the electrodes 15, may be placed on a subject.

In this embodiment, for the device for acquiring brain electrical signal 100, acquiring the cortical EEG signals is implemented by the plurality of electrodes 15 and the electrode base 2. The amplifier is arranged inside the electrode base 2, and then the amplifier transmits the cortical EEG signals acquired by the plurality of electrodes 15 arranged on the electrode base 2 to a host computer through a wired or wireless transmission path.

In an embodiment, referring to FIG. 3, in the device for acquiring brain electrical signal 100, each of the electrodes 15 has a first end and a second end. The first end of the electrode 15 is a physiological signal monitoring end. The first end of the electrode 15 is configured to be inserted into a living body to monitor the physiological signals. The second end of the electrode 15 is provided with a spiral wire 14. The second end of the electrode 15 is connected to the electrode base 2 by means of the spiral wire 14. A length of the electrode 15 is not limited and may be designed according to actual requirements. The electrode 15 is fixedly connected to the screw hole 13 on the electrode base 2 by means of the spiral wire 14.

In some embodiments, the electrode 15 is an electrode needle. Specifically, the electrode 15 is a threaded electrode needle. The electrode 15 (electrode needle) is inserted into the skull of the living body to monitor the physiological signals. The physiological signal monitored by the electrode needle is exported to the physiological signal monitoring device by an amplifier arranged in the electrode base 2. Specifically, the amplifier may be installed on the electrode base 2.

In an embodiment, in the device for acquiring brain electrical signal 100, the plurality of screw holes 13 are arranged in an array on the electrode base 2. The number of the plurality of screw holes 13 is not limited, and may be determined according to the number of the plurality of electrodes 15. When the plurality of screw holes 13 are arranged in the array, the distance therebetween is not limited and may be adjusted according to the number of the screw holes 13.

In an embodiment, the device for acquiring brain electrical signal 100 further includes an amplitude regulating rod 4. The amplitude regulating rod 4 is connected to the electrode base 2 and configured to adjust a vibration amplitude. For example, when inserted into the living body and when monitoring the physiological signals, the device for acquiring brain electrical signal 100 needs to vibrate at different vibration amplitudes. When acquiring brain electrical signals at different positions, the device for acquiring brain electrical signal 100 also needs to vibrate at different vibration amplitudes. Therefore, the amplitude regulating rod 4 is needed to continuously adjust the vibration amplitudes of the electrode 15.

In an embodiment, in the device for acquiring brain electrical signal 100, a surface of the electrode base 2 away from the amplitude regulating rod 4 is configured to be a round-edged surface 1. In this embodiment, the surface of the electrode base 2 away from the amplitude regulating rod 4 is configured to be the round-edged surface 1, which enables the electrode base 2 to be inserted into the living body more smoothly, thereby avoiding damages to skin.

In an embodiment, the device for acquiring brain electrical signal 100 further includes water pore 3. The water pore 3 is disposed in the amplitude regulating rod 4. The water pore 3 is configured to lower a temperature in a process of drilling. In this embodiment, the arrangement of the water pore 3 prevents the device for acquiring brain electrical signal 100 from damaging the living body due to a temperature increase during use, or avoids shortening a service life of the device for acquiring brain electrical signal 100 due to a temperature increase thereof.

In an embodiment, the device for acquiring brain electrical signal 100 further includes a fixing case 5. The fixing case 5 is connected to the amplitude regulating rod 4. The plurality of electrodes 15 are fixed on the fixing case 5. The fixing case 5 is configured to fix the plurality of electrodes and to provide a handle end.

In an embodiment, the device for acquiring brain electrical signal 100 further includes a transducer unit 20. The transducer unit 20 is electrically connected to the plurality of electrodes 15 and configured to supply energy to the plurality of electrodes 15. The transducer unit 20 is configured to generate mechanical waves, namely, ultrasound. The device for acquiring brain electrical signal 100 provided by this application may vibrate at a frequency of 20 kHz to 50 kHz, and removes biological tissues based on a mechanical effect, a cavitation effect, and a thermal effect of ultrasonic waves.

Further, referring to FIG. 1, the transducer unit 20 includes a front cap 6, piezoelectric ceramics 7, electrode plates 8, a first electrode wire 9, a second electrode wire 10, and a rear cap 11. The piezoelectric ceramics 7 are arranged between the front cap 6 and the rear cap 11. A plurality of piezoelectric ceramics 7 are provided, and the piezoelectric ceramics 7 are connected in series in the mechanical structure and connected in parallel in the circuit structure. Vibrations may be generated when an alternating current flows through the piezoelectric ceramics 7. A plurality of electrodes 8 are provided between the front cap 6 and the rear cap 11. The plurality of electrodes 8 and the plurality of piezoelectric ceramics 7 are electrically connected one-to-one. The electrode plates 8 supply power to the piezoelectric ceramics 7. The first electrode wire 9 is configured to be a positive electrode wire, and the second electrode wire 10 is configured to be a negative electrode wire. Alternatively, the first electrode wire 9 is configured to be the negative electrode wire, and the second electrode wire 10 is configured to be the positive electrode wire. Two ends of each electrode plate 8 are electrically connected to the first electrode wire 9 and the second electrode wire 10, respectively. The transducer unit 20 further includes a central shaft 12. The plurality of electrodes 15 are received inside the central shaft 12. The first end of the electrode 15 is an electrode needle. The electrode needle is inserted into the skull of the living body to monitor the physiological signals. The physiological signals monitored by the electrode needle are exported to the physiological signal monitoring device by means of the amplifier arranged on the electrode base 2. The central shaft 12 is configured to run to a position of the spiral wire 14 provided at the second end of the electrode 15.

The transducer unit 20 in this application includes a transducer. The transducer may be the piezoelectric ceramics 7 in the embodiments of the present application or other transducers, which is not further limited herein.

In an embodiment, the fixing case 5, the amplitude regulating rod 4, the electrode base 2 and the rear cap 11 are all insulated. The insulating material may be at least one of polyurethane, silicone resin, polytetrafluoroethylene, fluoropolymer, parylene, and polyimide.

The device for acquiring brain electrical signal 100 provided by this application is used for acquiring cortical EEG. Specifically, the device for acquiring brain electrical signal 100 provided by this application may be obtained by making improvements based on a traditional ultrasonic osteotome, and is used for acquiring cortical EEG.

The device for acquiring brain electrical signal 100 provided by this application monitors the physiological signals of the living body, or the device for acquiring brain electrical signal 100 acquires brain electrical signals, and it has the following technical advantages: it is convenient and efficient and takes a short time to monitor the physiological signals of the living body; by means of frequency adjustment, no damages are caused to the soft tissues when the bone is drilled through. Compared with the brain electrical signals, such as EEG, acquired by the existing non-intrusive electrophysiological monitoring method, the brain electrical signals acquired by the device for acquiring brain electrical signal 100 have higher quality, and better robustness and stability of anti-noise ability. Compared with the existing intrusive/semi-intrusive electrophysiological monitoring method such as ECoG, there is an extremely small risk to have a surgery of acquiring the brain electrical signals by using the device for acquiring brain electrical signal 100, and there is a relatively small possibility of accidents caused by unskilled operation in the surgery. In addition, the device for acquiring brain electrical signal 100 may also include a pressure sensor and a stopper, so that an automatic implantation surgery may be achieved completely, which is very beneficial to generalization.

From another point of view (in principle), the device for acquiring brain electrical signal 100 uses high-intensity focused ultrasound technology, converts electrical energy into mechanical energy by means of the transducer, and by means of high-frequency ultrasonic vibrations, makes intracellular water in the contacted tissues vaporize and protein hydrogen bonds break, thereby completely destroying the bone tissues that needs to be cut during the surgery. When the device for acquiring brain electrical signal 100 is in use, a temperature of the plurality of electrodes 15 is lower than 38° C., and the surrounding propagation distance is less than 200 microns. The high-intensity focused ultrasound has a destructive effect only on the bone tissue with a certain hardness, therefore not only will it not damage the blood vessels and nerve tissues, but it will enable the surgical wound to stop bleeding, thereby further reducing the wound of a minimally intrusive surgery, and greatly improving the accuracy, reliability and safety of the surgery. The device for acquiring brain electrical signal 100 acquires and records the physiological signals in real time by means of the plurality of electrodes 15. The device for acquiring brain electrical signal 100 may detect and record by using a professional instrument that records currents released by tissues such as nerves and muscles.

The present application also provides a method for acquiring brain electrical signal, which includes monitoring physiological signals, by the electrodes 15 of any device for acquiring brain electrical signal 100 described above. In an embodiment, only the device for acquiring brain electrical signal 100 is used for drilling, and the electrodes 15 are removed after a drilling is completed. Because the physiological saline used for cooling and the tissue fluid of the living body are conductive, the purpose of breaking through the non-conductive tissue of the skull may be achieved even if the plurality of electrodes 15 are directly removed.

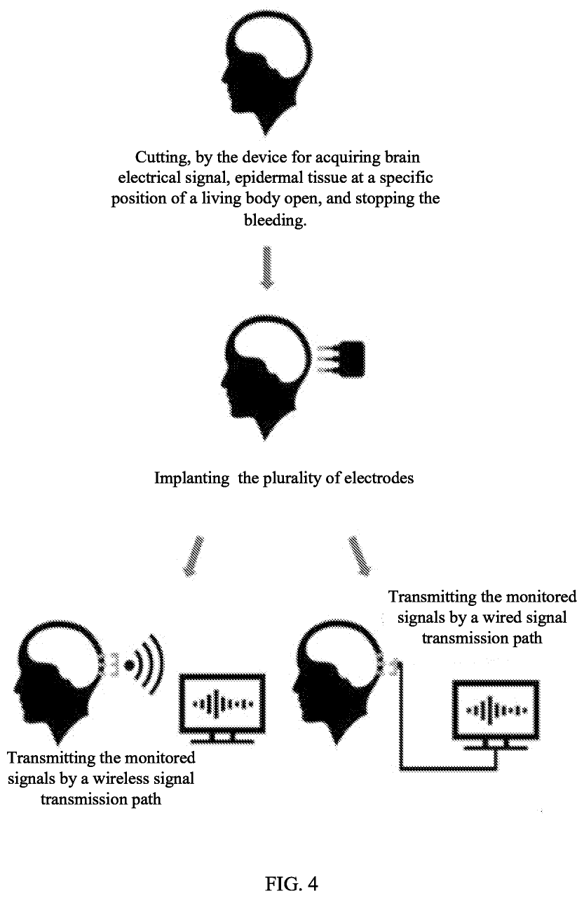

Please refer to FIG. 4, FIG. 4 illustrates a schematic flow chart of steps of the method for acquiring brain electrical signal. Specific steps for monitoring cortical EEG physiological signals may include following steps.

In step S10, epidermal tissue at a specific position of the living body is cut open by the device for acquiring brain electrical signal 100 and bleeding is stopped. This step may also be implemented by using devices such as an ultrasonic bone knife, an electrosurgical knife, a laser knife, a scalpel, or LEEP knife.

In step S20, the plurality of electrodes 15 or electrodes of an array in the device for acquiring brain electrical signal 100 are implanted into the living body.

In step S30, the cortical EEG physiological signals are displayed by a monitor after being transmitted by a wireless signal transmission path or a wired signal transmission path.

The method for acquiring brain electrical signal provided by this application uses the aforementioned device for acquiring brain electrical signal 100 to acquire the cortical EEG signals of the living body. Specifically, when monitoring physiological of the living body, the method for acquiring brain electrical signal provided by this application has the following technical advantages: it is convenient and efficient and takes a short time to monitor the physiological signals of the living body; by means of frequency adjustment, no damages are caused to the soft tissues when the bone is drilled through. Compared with the brain electrical signals such as EEG acquired by the existing non-intrusive electrophysiological monitoring method, the brain electrical signals acquired by the device for acquiring brain electrical signal 100 have higher quality, and better robustness and stability of anti-noise ability. Compared with the existing intrusive/semi-intrusive electrophysiological monitoring method such as ECoG, there is an extremely small risk to have a surgery of acquiring the brain electrical signals by using the device for acquiring brain electrical signal 100, and there is a relatively small possibility of accidents caused by unskilled operation in the surgery. In addition, the device for acquiring brain electrical signal 100 may also include a pressure sensor and a stopper, so that an automatic implantation surgery may be achieved completely, which is very beneficial to generalization.

Various technical features of the above embodiments may be arbitrarily combined. In order to simplify the description, not all possible combinations of the various technical features in the embodiments described above are described. However, as long as the combinations of these technical features are not contradictory, they should be considered within the scope described in this disclosure.

The embodiments described above are only several implementations of the present application, and the description of which is more specific and detailed, but cannot therefore be understood to be limitation of the scope of the present disclosure. It should be noted that, for those of ordinary skill in the art, various modifications and improvements may be made without departing from the concept of the present application, and all of the modifications and improvements fall within the protection scope of the present application. Therefore, the protection scope of the present application shall be subject to the appended claims.

What is claimed is:

1. A device for acquiring brain electrical signal, comprising:
a plurality of electrodes; and
an electrode base, provided with a plurality of screw holes;
wherein each of the plurality of electrodes passes through a corresponding screw hole of the plurality of screw holes.

2. The device for acquiring brain electrical signal according to claim 1, further comprising:
an amplifier arranged on the electrode base.

3. The device for acquiring brain electrical signal according to claim 2, wherein:
each of the plurality of electrodes has a first end and a second end; the first end of each of the plurality of electrode is a physiological signal monitoring end; the second end of each of the plurality of electrode is provided with a spiral wire; and the second end of each of the plurality of electrode is connected to the electrode base by the spiral wire.

4. The device for acquiring brain electrical signal according to claim 1, wherein the plurality of screw holes are arranged in an array on the electrode base.

5. The device for acquiring brain electrical signal according to claim 1, further comprising an amplitude regulating rod, connected to the electrode base and configured to adjust a vibration amplitude.

6. The device for acquiring brain electrical signal according to claim 5, wherein a surface of the electrode base away from the amplitude regulating rod is a round-edged surface.

7. The device for acquiring brain electrical signal according to claim 5, further comprising a water pore, disposed in the amplitude regulating rod.

8. The device for acquiring brain electrical signal according to claim 5, further comprising a fixing case, connected to the amplitude regulating rod, wherein the plurality of electrodes are fixed on the fixing case.

9. The device for acquiring brain electrical signal according to claim 8, further comprising a transducer unit, electrically connected to the plurality of electrodes, and configured to supply energy to the plurality of electrodes.

10. The device for acquiring brain electrical signal according to claim 1, wherein each of the plurality of the electrodes is a threaded electrode needle.

11. The device for acquiring brain electrical signal according to claim 2, wherein the amplifier is arranged inside the electrode base or on an outer side wall of the electrode base.

12. The device for acquiring brain electrical signal according to claim 9, wherein:
the transducer unit comprises a front cap, a plurality of piezoelectric ceramics, a plurality of electrodes, a first electrode wire, a second electrode wire, and a rear cap;
the plurality of electrodes and the plurality of piezoelectric ceramics are arranged between the front cap and the rear cap;
the plurality of electrodes and the plurality of piezoelectric ceramics are electrically connected one-to-one; and
two ends of each of the plurality of electrodes are electrically connected to the first electrode wire and the second electrode wire, respectively.

13. The device for acquiring brain electrical signal according to claim 9, wherein:
the transducer unit further comprises a central shaft;
the plurality of electrodes are received inside the central shaft; the central shaft is configured to run to a position of a spiral wire arranged at the second end of each of the plurality of electrodes; and
the first end of each of the plurality of electrodes is an electrode needle.

14. The device for acquiring brain electrical signal according to claim 12, wherein the fixing case, the amplitude regulating rod, the electrode base and the rear cap are made of insulating material.

15. The device for acquiring brain electrical signal according to claim 14, wherein the insulating material is at least one of polyurethane, silicone resin, polytetrafluoroethylene, fluoropolymer, parylene, and polyimide.

16. The device for acquiring brain electrical signal according to claim 1, wherein a material of the plurality of electrodes is at least one of metal, metal alloy, and conductive polymer.

17. The device for acquiring brain electrical signal according to claim 1, further comprising a pressure sensor and a stopper.

18. A method for acquiring brain electrical signal, comprising: monitoring physiological signals by using the device for acquiring brain electrical signal according to claim 1.

19. The method for acquiring brain electrical signal of claim 18, wherein, the monitoring physiological signals by using the device for acquiring brain electrical signal comprises:
cutting, by the device for acquiring brain electrical signal, epidermal tissue at a specific position of a living body open, and stopping bleeding;
implanting the plurality of electrodes or electrodes of an array in the device for acquiring brain electrical signal into the living body;
transmitting, by a wireless signal transmission path or a wired signal transmission path, cortical electroencephalogram physiological signals; and
displaying, by a monitor, transmitted cortical electroencephalogram physiological signals.
